(19) United States
(12) Patent Application Publication
(10) Pub. No.: US 2023/0226030 A1
(43) Pub. Date: Jul. 20, 2023

(54) THERAPEUTIC COMBINATIONS COMPRISING A RAF INHIBITOR FOR USE IN TREATING BRAF MUTANT NSCLC

(71) Applicant: Novartis AG, Basel (CH)

(72) Inventors: Giordano CAPONIGRO, Cambridge, MA (US); Uz Martin STAMMBERGER, Weiler b. Bingen (DE)

(21) Appl. No.: 17/760,298

(22) PCT Filed: Feb. 17, 2021
(86) PCT No.: PCT/IB2021/051336

§ 371 Date: Aug. 5, 2022

Related U.S. Application Data
(60) Provisional application No. 63/117,382, filed on Nov. 23, 2020.
(60) Provisional application No. 62/978,140, filed on Feb. 18, 2020.

(51) Int. Cl. A61K 31/444; A61K 31/519; A61P 35/00; A61K 9/00

(52) U.S. Cl. CPC A61K 31/444; A61K 31/519; A61P 35/00; A61K 9/0053

(57) ABSTRACT

The present invention provides a pharmaceutical combination comprising a CRAF inhibitor plus (i) an ERK inhibitor or (ii) a MEK inhibitor, for use in the treatment of non-small cell lung cancer (NSCLC) where the carcinoma cells of the NSCLC harbor a mutation of BRAF other than the BRAF V600E-mutant, and related invention aspects. The present invention also provides dosages and dosage regimens of a CRAF inhibitor with an ERK inhibitor or with trametinib for use in the treatment of BRAF V600 mutant (e.g. BRAF V600E mutant) NSCLC.

### RELATED APPLICATIONS

This application is a § 371 U.S. National Stage Entry of International Application No. PCT/IB2021/051336, filed Feb. 17, 2021, which claims the benefit of U.S. Provisional Application Ser. No. 63/117,382, filed Nov. 23, 2020, and U.S. Provisional Application Ser. No. 62/978,140, filed Feb. 18, 2020, each of which are hereby incorporated by reference in their entirety.

### FIELD OF THE INVENTION

The present invention relates to the use of a RAF inhibitor, particularly an inhibitor of CRAF, especially N-(3-(2-(2-hydroxy ethoxy)-6-morpholinopyridin-4-yl)-4-methylphenyl)-2-(trifluoromethyl)isonicotinamide (Compound A below) in combination with (a) an ERK inhibitor (ERKi), especially 4-(3-amino-6-((1S,3 S,4S)-3-fluoro-4-hydroxycydohexyl)pyrazin-2-yl)-N-((S)-1-(3-bromo-5-fluorophenyl)-2-(methylamino)ethyl)-2- fluorobenzamide (Compound B below) or (b) trametinib (where in each case the inhibitor may be independently selected from the free compound or a salt or solvate thereof) for use in the treatment of lung cancer, especially Non-Small Cell Lung Cancer (NSCLC). It particularly relates to therapeutic combinations using at least one RAF (e.g., a CRAF) inhibitor and at least one ERK inhibitor for the treatment of

NSCLC. The invention also relates to a pharmaceutical combination which comprises (a) at least one ERK inhibitor, and (b) at least one RAF inhibitor that is preferably a CRAF inhibitor that may also inhibit BRAF, where the two compounds are prepared and/or used (or for use) for simultaneous, separate or sequential administration for the treatment of said lung cancer, and to a pharmaceutical composition comprising such combination; a method of treating a subject having said lung cancer comprising administration of said combination to a subject in need thereof; use of such combination for the treatment of said lung cancer; and a commercial package comprising such combination.

In the present invention, the lung cancer to be treated is preferably non-small lung cancer (NSCLC), in particular, BRAF non-V600 mutant NSCLC (e.g. BRAF non-V600E mutant NSCLC) or a Class I, II or III BRAF-mutantNSCLC (more particularly, NSCLC with Class II BRAF mutations or NSCLC with class III BRAF mutations). In certain aspects of the invention, said lung cancer is also a NSCLC (non-small cell lung cancer) where the carcinoma cells of the NSCLC harbor a mutation of BRAF other than the BRAF V600E-mutation. Typically, the CRAF inhibitor and the ERK inhibitor are both low-molecular weight compounds, and in particular the invention relates to a combination of Compound A and Compound B or a combination of Compound A and trametinib defined below for use as described herein.

In particular, there is provided (i) a pharmaceutical combination comprising Compound A, or a pharmaceutically acceptable salt thereof, and Compound B, or a pharmaceutically acceptable salt thereof, or (ii) a pharmaceutical combination comprising Compound A, or a pharmaceutically acceptable salt thereof, and trametinib or a pharmaceutically acceptable salt or solvate thereof, for use in the treatment of BRAF non-V600 (e.g. non-V600E) NSCLC, i.e. NSCLC (non-small cell lung cancer) where the carcinoma cells of the NSCLC harbor a mutation of BRAF other than BRAF V600 (e.g. non-V600E) mutations. These pharmaceutical combinations may be particularly useful in the treatment of BRAF mutant NSCLC, including advanced or metastatic BRAF mutant NSCLC.

In another aspect, the present invention relates to specific doses and dosing regimens in a combination therapy employing Compound A, or pharmaceutically acceptable salt thereof, and Compound B, or pharmaceutically acceptable salt thereof, which may be particularly useful for the treatment of BRAF V600 mutant (e.g. BRAF V600E) mutant NSCLC), e.g. wherein the treatment is characterized by increase of tolerability and/or maintenance of anti-tumor activity.

In yet another aspect, the present invention relates to specific doses and dosing regimens in a combination therapy employing Compound A, or pharmaceutically acceptable salt thereof, and trametinib, or pharmaceutically acceptable salt or solvate thereof, which may be particularly useful for the treatment of BRAF V600 mutant (e.g. BRAF V600E) mutant NSCLC), e.g. wherein the treatment is characterized by increase of tolerability and/or maintenance of anti-tumor activity.

### BACKGROUND OF THE INVENTION

The RAS/RAF/MEK/ERK or MAPK pathway is a key signaling cascade that drives cell proliferation, differentiation, and survival. Dysregulation of this pathway underlies many instances of tumorigenesis. Aberrant signaling or inappropriate activation of the MAPK pathway has been shown in multiple tumor types, including melanoma, lung and pancreatic cancer, and can occur through several distinct mechanisms, including activating mutations in RAS and BRAF. RAS is a superfamily of GTPases, and includes KRAS (v-Ki-ras2, Kirsten rat sarcoma viral oncogene homolog), which is a regulated signaling protein that can be turned on (activated) by various single-point mutations, which are known as gain of function mutations. The MAPK pathway is frequently mutated in human cancer with KRAS and BRAF mutations being among the most frequent (approximately 30%). RAS mutations, particularly gain of function mutations, have been detected in 9-30% of all cancers, with KRAS mutations (e.g., G12D, G12V or G12C) having the highest prevalence (86%), followed by NRAS (11%), and, infrequently, HRAS (3%) (Cox AD, Fesik SW, Kimmelman AC, et al (2014), Nat Rev Drug Discov. Nov; 13(11):828-51.).

Emerging evidence on the role of CRAF in mediating KRAS signaling and in the development of KRAS-mutant non-small cell lung cancer (NSCLC) makes it a suitable target for therapeutic intervention (Blasco RB, Francoz S, Santamaria D, et al (2011) CRAF, but not B-Raf, is essential for development of K-Ras oncogene-driven non-small cell lung carcinoma. Cancer Cell. 2011 May 17;19(5):652-63.). CRAF was shown to promote feedback-mediated pathway reactivation following MEK inhibitor treatment in KRAS-mutant cancers (Lito P, Saborowski A, Yue J, et al (2014) Disruption of CRAF-MediatedMEK Activation Is Required for Effective MEK Inhibition in KRAS Mutant Tumors. Cancer Cell 25, 697-710., Lamb a et al 2014). In addition, CRAF plays an essential role in mediating paradoxical activation following BRAF inhibitor treatment (Poulikakos PI, Zhang C, Bollag G, et al. (2010), Nature. Mar 18; 464(7287):427-30., Hatzivassiliou et al 2010, Heidorn et al 2010). Thus, selective pan-RAF inhibitors that potently inhibit the activity of CRAF and BRAF could be effective in blocking BRAF-mutant tumors and RAS-mutant driven tumorigenesis and may also alleviate feedback activation. Compound A described herein is a potent inhibitor of both CRAF and BRAF.

Lung cancer is a common type of cancer that affects men and women around the globe. NSCLC is the most common type (roughly 85%) of lung cancer with approximately 70% of these patients presenting with advanced disease (Stage IIIB or Stage IV) at the time of diagnosis.

Mutations leading to lung cancer can be found on MEK1, PIK3CA, NTRK, RET, HER2, MET, ROS1, ALK, EGFR and others.

BRAF mutations have also been observed in up to 3-4% of NSCLC and have been described as a resistance mechanism in EGFR mutation positive NSCLC. About 50% of the BRAF mutations in NSCLC is a valine to glutamic acid substitution at codon 600 (V600E).

BRAF V600E is constitutively active and signals downstream via MEK-ERK1/2 to promote cellular transformation independent of RAS binding and RAF dimerization.

BRAF mutations have also been classified into three classes. Class I BRAF mutations affect amino acid V600 and signal as RAS-independent active monomers, class II mutations function as RAS-independent activated dimers, and class III mutations are kinase impaired but increase signaling through the MAPK pathway due to enhanced RAS binding and subsequent CRAF activation (see M. Dankner et al., Oncogene (2018) 37, 3183-3199, which is hereby incorporated by reference in its entirety, especially with regard to definition of the classes, mutant NSCLC cell lines useful in determining the efficiency of inhibitors and possible co-inhibitors).

Whereas for the subgroup of patients suffering from BRAF V600E NSCLC, a targeted combination, consisting of the BRAF inhibitor dabrafenib and the MEK inhibitor trametinib, has been approved, no targeted therapy is currently available for a large subgroup of NSCLC patients with non-V600E BRAF mutations (see Dagogo-Jack et al 2019, Clin Cancer Res 25(1) January 1, 2019, 158-165, which is hereby also incorporated by reference in its entirety, and Dankner et al, 2018). Furthermore, the outcome to standard of care chemotherapies (e.g. platinum-based chemotherapy) seems to be less efficacious for these non-V600 BRAF mutated NSCLC patients as compared to both BRAF V600 mutant NSCLC or NSCLC patients lacking any driver mutations, thus defining non-V600 BRAF mutations as a negative prognostic factor.

To date, there are no approved targeted therapies for NSCLC patients with KRAS mutations and BRAF mutations other than V600E.

There is thus a high unmet medical need for patients suffering from atypical BRAF mutant NSCLC, or from BRAF non-V600 mutant (e.g. BRAF non-V600E mutant) NSCL or from a Class I, II or III BRAF-mutantNSCLC (more particularly, NSCLC with Class II BRAF mutations or NSCLC with class III BRAF mutations).

There is a need for targeted therapy that is safe and/or well tolerated. A therapy which results in durable and sustained responses in a clinical setting would also be beneficial.

### SUMMARY OF THE INVENTION

The present invention especially provides a pharmaceutical combination which comprises:

a CRAF inhibitor which is Compound A,

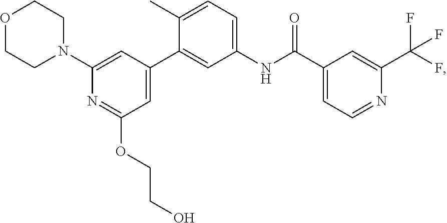 [SMILES: Cc1ccc(NC(=O)c2ccnc(C(F)(F)F)c2)cc1-c1cc(OCCO)nc(N2CCOCC2)c1]

or a pharmaceutically acceptable salt thereof, and
(i) an ERK inhibitor which is Compound B,

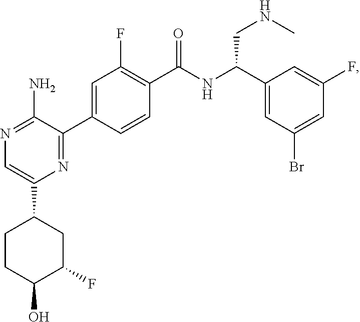 [SMILES: CNC[C@@H](NC(=O)c1ccc(-c2nc([C@H]3CC[C@H](O)[C@@H](F)C3)cnc2N)cc1F)c1cc(F)cc(Br)c1]

or a pharmaceutically acceptable salt thereof, especially the HCl salt, or
(ii) an MEK inhibitor which is trametinib,

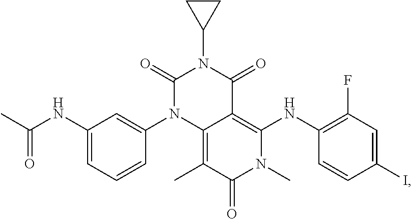 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate (e.g. the dimethyl sulfoxide solvate) thereof.

These combinations are also referred herein as the “combination of the invention”.

The present invention provides:

A combination of the invention for use in the treatment of atypical BRAF mutant NSCLC;

A combination of the invention for use in the treatment of BRAF non-V600 mutant

NSCLC (e.g. BRAF non-V600E mutant NSCLC) or of a Class I, II or III BRAF-mutant

NSCLC (more particularly, NSCLC with Class II BRAF mutations or NSCLC with class III BRAF mutations);

Compound A, or a pharmaceutically acceptable salt thereof, for use in the treatment of

BRAF non-V600 mutant NSCLC (e.g. BRAF non-V600E mutantNSCLC) or of a Class I, II or III BRAF-mutant NSCLC (more particularly, NSCLC with Class II BRAF mutations or NSCLC with class III BRAF mutations), wherein Compound A is co-administered with an ERK inhibitor such as Compound B, or a pharmaceutically acceptable salt (e.g. the hydrochloride salt) thereof; optionally wherein the total daily dose (TTD) of Compound A is administered twice daily (BID), e.g. from 200 mg BID to 400 mg BID, and Compound B is administered in a total daily dose (TTD) from 100 mg to 400 mg (typically from 100 mg to 200 mg (preferably administered once daily (QD));

Compound A, or a pharmaceutically acceptable salt thereof, for use in the treatment of BRAF non-V600 mutant NSCLC (e.g. BRAF non-V600E mutantNSCLC) or of a Class I, II or III BRAF-mutant NSCLC (more particularly, NSCLC with Class II BRAF mutations or NSCLC with class III BRAF mutations), wherein Compound A is co-administered with trametinib, or a pharmaceutically acceptable salt or solvate (e.g. the dimethyl sulfoxide solvate) thereof; optionally wherein the total daily dose (TTD) of Compound A is administered twice daily, e.g. from 200 mg BID to 400 mg BID, and trametinib is administered in a total daily dose (TTD) from 0.5 mg to 2 mg (typically from 0.5 mg to 1.0 mg (preferably administered once daily));

Compound B, or a pharmaceutically acceptable salt (e.g. the hydrochloride salt) thereof, for use in the treatment of BRAF non-V600 mutant NSCLC (e.g. BRAF non-V600E mutant NSCLC) or of a Class I, II or III BRAF-mutantNSCLC (more particularly, NSCLC with Class II BRAF mutations or NSCLC with class III BRAF mutations); wherein Compound B is co-administered with Compound A, or a pharmaceutically acceptable salt thereof; optionally wherein the total daily dose (TTD) of Compound A is administered twice daily (BID), e.g. from 200 mg BID to 400 mg BID, and Compound B is administered in a total daily dose (TTD) from 100 mg to 400 mg (typically from 100 mg to 200 mg (preferably administered once daily (QD));

trametinib, ora pharmaceutically acceptable salt or solvate (e.g. the dimethyl sulfoxide solvate) thereof, for use in the treatment of BRAF non -V600 mutant NSCLC (e.g. BRAF non-V600E mutant NSCLC) or of a Class I, II or III BRAF-mutantNSCLC (more particularly, NSCLC with Class II BRAF mutations or NSCLC with class III BRAF mutations), wherein trametinib is co-administered with Compound A, or a pharmaceutically acceptable salt thereof; optionally wherein the total daily dose (TTD) of Compound A is administered twice daily, e.g. from 200 mg BID to 400 mg BID, and trametinib is administered in a total daily dose (TTD) from 0.5 mg to 2 mg (typically from 0.5 mg to 1.0 mg (preferably administered once daily));

Compound A, or a pharmaceutically acceptable salt thereof, for use in the treatment of BRAF V600 mutant NSCLC (e.g. BRAF V600E mutantNSCLC), wherein Compound A is co-administered with Compound B, or a pharmaceutically acceptable salt thereof, and Compound A is administered in a total daily dose (TTD) from 400 mg to 800 mg (preferably wherein the TTD of Compound A is administered twice daily (BID), e.g. from 200 mg BID to 400 mg BID) and Compound B is administered in a total daily dose (TTD) from 100 mg to 400 mg (typically from 100 mg to 200 mg (preferably administered once daily (QD));

Compound B, or a pharmaceutically acceptable salt thereof, for use in the treatment of BRAF V600 mutant NSCLC (e.g. BRAF V600E mutantNSCLC), wherein Compound B is co-administered with Compound A, or a pharmaceutically acceptable salt thereof, and Compound B is administered in a total daily dose (TTD) from 100 mg to 400 mg (typically from 100 mg to 200 mg (preferably administered once daily)) and Compound A is administered in a total daily dose (TTD) from 400 mg to 800 mg (preferably wherein the TTD of Compound A is administered twice daily, e.g. from 200 mg BID to 400 mg BID);

Compound A, or a pharmaceutically acceptable salt thereof, for use in the treatment of BRAF V600 mutant NSCLC (e.g. BRAF V600E mutant NSCLC), wherein Compound A is co-administered with trametinib, or a pharmaceutically acceptable salt or solvate thereof, wherein Compound A is administered in a total daily dose (TTD) from 400 mg to 800 mg (preferably wherein the TTD is administered twice daily, e.g. from 200 mg BID to 400 mg BID) and trametinib is administered in a total daily dose (TTD) from 0.5 mg to 2 mg (typically from 0.5 mg to 1.0 mg (preferably administered once daily));

trametinib, or a pharmaceutically acceptable salt or solvate (e.g. the dimethyl sulfoxide solvate) thereof, for use in the treatment of BRAF V600 mutant NSCLC (e.g. BRAF

V600E mutant NSCLC), wherein Compound A is co-administered with trametinib (trametinib), or a pharmaceutically acceptable salt or solvate thereof, wherein Compound A is administered in a total daily dose (TTD) from 400 mg to 800 mg (preferably wherein the TTD is administered twice daily, e.g. from 200 mg BID to 400 mg BID) and trametinib is administered in a TTD from 0.5 mg to 2 mg (typically from 0.5 mg to 1.0 mg (preferably administered once daily)).

The present invention further provides a pharmaceutical combination comprising a CRAF kinase inhibitor, which is Compound A, or a pharmaceutically acceptable salt thereof, and an ERK inhibitor, which is Compound B, or a pharmaceutically acceptable salt thereof, as described herein, for simultaneous, separate or sequential (in any order) administration, for use in the treatment of NSCLC (non-small cell lung cancer) with a BRAF non-V600 mutation i.e. where the carcinoma cells of the NSCLC harbor a mutation of BRAF other than the BRAF V600E-mutation. The present invention is thus related to the combination of the invention for use in the treatment of a proliferative disease characterized by activating mutations in the MAPK pathway, and in particular by one or more mutations in BRAF. The present invention is particularly related to the treatment of BRAF-mutant NSCLC where the carcinoma cells of the

NSCLC harbor a mutation of BRAF other than the BRAF V600E-mutant.

The present invention thus provides a pharmaceutical combination as described herein wherein the treatment is safe and/or well tolerated, or which provides a treatment which results in durable and sustained responses in patients suffering from BRAF-mutant NSCLC, particularly, BRAF non-V600 mutant NSCLC (e.g. BRAF non-V600E mutant NSCLC) or of a Class I, II or III BRAF-mutant NSCLC (more particularly, NSCLC with Class II BRAF mutations or NSCLC with class III BRAF mutations).

Compound A is an adenosine triphosphate (ATP)-competitive inhibitor of BRAF (also referred to herein as b-RAF or BRAF) and CRAF (also referred to herein as CRAF) protein kinases. Throughout the present invention, Compound A is also referred to as a CRAF inhibitor or a CRAF kinase inhibitor.

Compound A is N-(3-(2-(2-hydroxyethoxy)-6-morpholinopyridin-4-yl)-4-methylphenyl)-2-(trifluoromethyl)isonicotinamide and is the compound of the following structure:

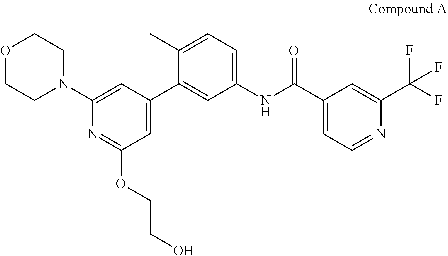 [SMILES: Cc1ccc(NC(=O)c2ccnc(C(F)(F)F)c2)cc1-c1cc(OCCO)nc(N2CCOCC2)c1]

Compound A is Example 1156 in published PCT application W02014/151616. The preparation of Compound A, pharmaceutically acceptable salts of Compound A and pharmaceutical compositions comprising compound A are also disclosed in the PCT application, e.g., see pages 739-741. Compound A is also known and referred to herein as “LXH254”.

In cell-based assays, Compound A has demonstrated anti-proliferative activity in cell lines that contain a variety of mutations that activate MAPK signaling. In vivo, treatment with Compound A generated tumor regression in several KRAS-mutant models including the NSCLC-derived Calu-6 (KRAS Q61K) and NCI-H358 (KRAS G12C). Collectively, the in vitro and in vivo MAPK-pathway suppression and anti-proliferative activity observed for Compound A at well-tolerated doses supports the assumption that Compound A may have anti-tumor activity in patients with tumors harboring activating lesions in the MAPK pathway. Compound A is a Type 2 ATP-competitive inhibitor of both B-Raf and CRAF that keeps the kinase pocket in an inactive conformation, thereby reducing the paradoxical activation seen with many B-Raf inhibitors, and blocking mutant RAS-driven signaling and cell proliferation. Compound A has exhibited efficacy in numerous MAPK-driven human cancer cell lines and in xenograft tumors representing model tumors harboring human lesions in KRAS, NRAS and BRAF oncogenes.

Compound B is an inhibitor of extracellular signal-regulated kinases 1 and 2 (ERK 1/2). Compound B is known by the name of 4-(3-amino-6-((1S,35,45)-3-fluoro-4-hydroxycyclohexyl)pyrazin-2-yl)-N-((S)-1-(3-bromo-5-fluorophenyl)-2-(methylamino)ethyl)-2- fluorobenzamide and is the compound of the following structure.

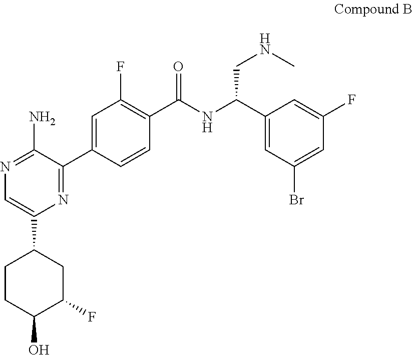 [SMILES: CNC[C@@H](NC(=O)c1ccc(-c2nc([C@H]3CC[C@H](O)[C@@H](F)C3)cnc2N)cc1F)c1cc(F)cc(Br)c1]

Compound B is disclosed and its preparation described in published PCT patent application W02015/066188. Compound B is also known as and referred to herein as “LTT462”.

Compound B has been shown to be active as a single-agent and combination therapy in models of various solid tumors.

The hydrochloride salt is a preferred example of a pharmaceutically acceptable salt of Compound B.

Trametinib is a potent and selective MEK 1/2 inhibitor. It is known by the name N-(3-{3-cyclopropyl-5-[(2-fluoro-4-iodophenyl)amino]-6, 8-dimethyl-2, 4,7-trioxo-3,4,6,7-tetrahydropyrido[4,3-d]pyrimidin-1(2H)-yl}phenyl)acetamide or trametinib and is the compound having the following structure:

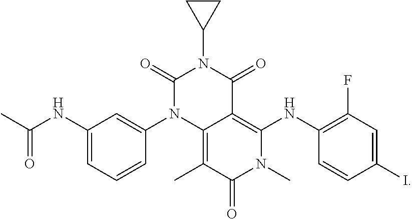 [SMILES: CC(=O)Nc1cccc(-n2c(=O)n(C3CC3)c(=O)c3c(Nc4ccc(I)cc4F)n(C)c(=O)c(C)c32)c1]

Trametinib (also known as MEKINIST®) is disclosed and its preparation described e.g., in W02005/121142, for example in Example 4-1 or in “Example 4-1 (alternative method)”.

Trametinib has been shown to be active in (especially metastatic) melanoma carrying the BRAF V600E mutation in a phase III clinical trial and is approved by the FDA for treatment of V600E mutated metastatic melanoma. Often resistance to single-agent was observed, which led to combination e.g., with the BRAF inhibitor dabrafenib in melanoma treatment.

Trametinib is approved in combination with dabrafenib for the treatment of patients with advanced NSCLC with a BRAF V600E mutation.

Reference to Compound A, Compound B or trametinib as used herein always refers to the free compounds and/or a salt thereof, unless context clearly dictates otherwise. An example of a salt of Compound B is the hydrochloride salt. Or in the case of trametinib, also the solvate, e.g., the dimethyl sulfoxide solvate thereof.

It was found that vertical MAPK (mitogen activated protein kinase) inhibition combining a dual BRAF and CRAF inhibitor such as Compound A with an ERK1/2 kinase inhibitor such as Compound B or with a MEK inhibitor such as trametinib optimizes suppression of MAPK signaling in BRAF mutant NSCLC. This combination may also help to prevent the emergence of resistance to the combination of BRAF and MEK (mitogen-activated protein kinase kinase) inhibitors in BRAF non-V600E mutant NSCLC as can be observed with the single agent.

Disclosed herein are pharmaceutical combinations which comprise a CRAF inhibitor, such as Compound A, or a pharmaceutically acceptable salt thereof, and (b) an ERK inhibitor such as Compound B, or a pharmaceutically acceptable salt thereof, or (c) a MEK inhibitor such as trametinib, or a pharmaceutically acceptable salt thereof, for simultaneous, separate or sequential administration for use in the treatment of NSCLC (non-small cell lung cancer) with a BRAF non-V600 mutation i.e. the treatment of BRAF-mutant NSCLC (non-small cell lung cancer) wherein the carcinoma cells are harboring a mutation other than BRAF V600, e.g., wherein the carcinoma cells are harboring a mutation other than BRAF V600E. Also disclosed are a pharmaceutical composition comprising such combinations; a method of treating a subject having a NSCLC where the carcinoma cells of the NSCLC harbor a mutation of BRAF other than a BRAF V600 mutant, or other than the BRAF V600E-mutant, comprising administration of such combination to a subject in need thereof; use of such combination for the treatment ofNSCLC where the carcinoma cells of the NSCLC harbor a mutation of BRAF other than the BRAF V600-mutant or other than the BRAF V600E-mutant; and a commercial package comprising such combination. Other features, objects, and advantages of the invention will be apparent from the description and drawings, and from the claims.

### BRIEF DESCRIPTION OF THE FIGURES

FIG. 1 shows a Western Blot demonstrating improved inhibition of MAPK signaling in a cell line harboring a class III BRAF mutation. Inhibition of signaling by combined low-dose Compound A and trametinib (Compound C) was significantly stronger that comparable single agent treatments. Similarly, pathway suppression by combined Compound A and Compound B exceeded that observed for either single agent.

FIG. 2 shows representative xenograft models treated with Compound A. Shown on the left of each panel represented are changes in tumor volume over time (days) and on the right of each panel changes in DUSP6 mRNA level after a single administration of Compound A for (A) the SK-MEL-30 (NRAS^{Q61K}, BRAF^{D287H}) and (B) the HEYA8 (KRAS^{G12D}, BRAF^{G464E}) models. Vehicle (Veh) treated animals are indicated with (3) and LXH254 treated animals with shown (0). WM-793 xenografts were treated with LXH254 at 50 mg/kg bid, all other models were treated with LXH254 100 mg/kg qd. T/C determinations were made at the time when control animals were sacrificed due to tumor size (1000- 1500 mm³). Significance (indicated by *) compared to the vehicle control group is reported unless otherwise stated.

### DETAILED DESCRIPTION OF THE INVENTION

The following definitions of more general terms or features of the invention can be used to replace one, more than one or all terms features of each invention embodiment, thus resulting in more specific invention embodiments which all form part of the invention.

Selected terms are defined below and throughout the application.

Receptor tyrosine kinase (RTK) usually mediate their activity via a number of proteins named GRB2, GAB1 and SOS1 via a protein named SHP2. This normally leads to active GTP-RAS formation in the presence of SOS1, which can be deactivated by the negative regulator NF1 to GDP-RAS. Normally, activated RAS (GTP RAS) leads to activation of RAF (e.g., BRAF), which then leads to MEK activation, which leads to ERK (MAPK3) activation. This chain is also known as the MAP kinase (or ERK) signaling pathway. The MAPK cascade is a signaling pathway that serves to transmit extracellular proliferative signals to the nucleus of receptive cells.

In the case of mutated BRAF (BRAF mut), the mutation may lead to unregulated activation of BRAF, which leads to activation of MEK and ERK and thus uncontrolled cell growth.

The term “atypical BRAF mutant NSCLC” is refers to NSCLC where the cancer cells do not have a BRAF V600, more particularly a BRAF V600E, mutation.

The term “BRAF non-V600-mutantNSCLC” (in short form “non V600 mutant” within the present invention) relates to any other type of mutation and thus mutant of BRAF, including other class I (that is, other V600 mutants other than V600E) BRAF mutants, especially V600K, V600D or the like, or preferably “atypical” (that is, non-V600) mutants, selected from class II BRAF mutants, class III BRAF mutants and, moreover, not yet classified mutants and/or BRAF amplification mutants, where in each case the BRAF expression is higher than in BRAF wild type (WT) cells (e.g., as measured by Western Blotting or according to Davies H, Bignell GR,

Cox C, et al. Mutations of the BRAF gene in human cancer. Nature 2002;417:949-54; note in this reference BRAF mutation V600E is called V599E, based on a different reference sequence of BRAF).

The term “atypical BRAF mutant NSCLC” is

In one aspect of the invention, the term “Class I BRAF mutation” refers to a Class I BRAF mutation which is not a BRAF V600 E mutation.

Class I BRAF mutants (=Type I variants) refers to cancer cells such as NSCLC cells which activate the pathway in a RAS independent manner often as a monomer. All the V600 variants, including V600E are class I variants and include especially V600D, V600K, V600R or moreover V600L. Class I BRAF mutations induce a strong activation of the BRAF kinase, independent of RAS signaling, in these mutants. The growth in these cells is inhibited by BRAF inhibitors (such as dabrafenib) and MEK inhibitors (such as trametinib), which especially in combination lead to lower MAPK signaling. These variants activate the MAPK pathway independently from active RAS, and signal as a monomer. It is this latter property that makes them sensitive to type 1.5 RAF inhibitors (BRAF inhibitors; e.g., dabrafenib, vemurafenib, encorafenib).

Class II BRAF mutants ((=Type II variants) (also known as Class II non-V600 BRAF mutants) refers to cancer cells such as NSCLC cells with a BRAF mutation resulting in intermediate to high BRAF activity and are essentially RAS independent. These variants activate signaling in a RAS independent manner, but do so as a dimer. Signaling as a dimer makes them largely refractory to inhibition by type 1.5 inhibitors. There are anecdotal reports of patients with putative type II mutations responding to BRAF inhibitor treatment, but these are uncommon and often the response is to combined type 1.5 BRAF+MEK inhibitor treatment (MEK inhibitors are not dependent on the type of RAF mutation). Examples of Class II BRAF mutations include, but are not limited to:

1. BRAF kinase domain fusions;
2. Specific point mutations such as some but not all G469 variants (especially G469V, G469A, G469L, G469R, or GV469S), K601 variants and some but not all G464 variants, especially G464V or moreover G464E or G464R. Other particular mutants comprise E586K, F595L, L597C, L597R, L597S, L595V, A598V, T599I, K601E, K601N, K601T, A727V;
3. Small indels in BRAF such as NVTAPA which move the ac-helix to an “in” conformation.

Examples of class II BRAF mutations include L597Q, L597R, G464V, G464A, G469A, G469V, G469R, G4695, K601E, K601N, K601T, E451Q, A712T and fusions.

Examples of class II BRAF mutations include G469V, G469A, G469L and G464V, which are preferred as class II mutants according to preferred embodiments of the present invention but are not to be regarded as limiting in more general embodiments of the invention.

Class III mutants (=Type III variants) refers to cancer cells such as NSCLC cells with a BRAF mutation where cell growth depends on RAS signaling, Class III mutations are kinase impaired or kinase dead but increase signaling through the MAPK pathway in a RAS-dependent manner via transactivation of, e.g., CRAF. These mutants are often partly or completely kinase impaired, and they co-operate with activated RAS to drive RAF dimer-dependent signaling. RAS can be activated via mutations, loss of NF-1/GAP proteins, or non-genetic activation of upstream RTKs. In addition to RAS activating mutations, Receptor Tyrosine Kinase (RTK) overexpression and/or NF1-loss-of-function (e.g., nonsense or missense) mutations may contribute. Such variants in particular include, but are not limited to: D287H, D287Y, F595, G596, G466 (e.g., G466V, G466R, G466E or G466A), 5467A, S467E, S467L, G469E, K483M,

N5811, N581 S, D594 such as D594A, D594E, D594G, D594H, D594N or D594V, G596A, G596D, G596F or G596R and the like.

Examples of class III BRAF mutations include G469E, G466V, G466E, G466A, N581S N581I, D594G, D594N and G596R.

The class III pathway can potentially be modulated pharmacologically with RTK inhibitors, pan-Raf inhibitors such as sorafenib or paradox breaking BRAF dimerization inhibitors.

Both class II and class III mutants (which are preferred variants for combination treatment according to the present invention) thus include and especially only contain mutations other than V600 mutations. Most mutants likely fall into one of these categories, but have not been formally tested. It is likely that some variants cannot signal on their own, but still get a boost from activated RAS. Different amino acid substitutions at the same position can create a type II or a type III depending on the substitution.

The term “not yet classified mutants” refers to NSCLC cells with other than Class I, Class II and Class III (especially missense) mutants, such as E26A, V130M, and D284E, which are preferred as not yet classified missense mutants according to preferred embodiments of the present invention but are not to be regarded as limiting in more general embodiments of the invention; especially other than missense mutations, such as deletions and fusions other than those of class II.

Highly preferred mutations include BRAF D287H, G464E and G 466 V.

Where a mutation in BRAF is mentioned in the present invention, the position is as defined (e.g., for V600E) in the (as of Feb. 10, 2020) most recent reference sequences NM_004333.6 and NP_004324.2 (Genbank, NCBI).

Where “moreover” is mentioned, the features or designations before this word are more preferred than those after this word.

Genetic assessment of BRAF mutations in patients can be conducted using SNaPshot or DFCI Oncopanel as described previously (Sholl LM, et al. JCI Insight 2016;1: e87062; Zheng Z, et al., Nat Med 2014;20:1479-84). The current iterations of both assays utilize next-generation sequencing, whereas earlier versions of SNaPshot relied on multiplex PCR. The current version of SNaPshot interrogates exons 11 and 15 of BRAF, exons 2-5 of KRAS and NRAS, and exons 1-58 of NFL Oncopanel detects alterations involving all exons of BRAF, KRAS, NRAS, and NF1.

Activity of the present combination against NSCLC can be experimentally evidenced in NSCLC cell lines such as (for class II) HI755 cells (G469A), H2087 cells (L597V) or (for class III) Cal12T cells, G466V); against wild type (WT) BRAF comprising cells such as H1437.

In the present invention, the term lung cancer or NSCLC includes a BRAF non-V600E mutant NSCLC or a Class I, II or III BRAF-mutant NSCLC.

In one embodiment, the invention features a method of treating (e.g., inhibiting, reducing ameliorating, or preventing) a NSCLC in a subject, and in particular a NSCLC as described herein. The method includes administering to a subject, in combination with a CRAF inhibitor, an ERK inhibitor; in particular embodiments, the CRAF inhibitor is Compound A, and the ERK inhibitor is Compound B and the MEK inhibitor is trametinib. Suitable dosages and administration schedules for using these compounds in such methods are described herein.

Included are tumors having BRAF mutations others than V600E, especially as defined elsewhere herein. The CRAF inhibitor for use in the methods, treatments and combinations disclosed herein is a potent inhibitor of at least CRAF, and optionally also of BRAF. In some embodiments, the CRAF inhibitor, or a pharmaceutically acceptable salt thereof, is administered orally. In some embodiments, the CRAF inhibitor is Compound A or a pharmaceutically acceptable salt thereof.

When describing a dosage or dose herein as ‘about’ a specified amount the actual dosage can vary by up to 10%, e.g., 5%, from the stated amount: this usage of ‘about’ recognizes that the precise amount in a given dose or dosage form may differ slightly from an intended amount for various reasons without materially affecting the in vivo effect of the administered compound. Specific dosages and doses may also be described without being preceded by the term “about”; the skilled person will recognize that the dosage be within an acceptable degree of error for the quantity measured as is customary in the art. The skilled person will understand that where a dose or dosage of a therapeutic compound is quoted herein, that amount refers to the amount of the therapeutic compound in its free form, as is customary in the art.

The unit dosage of the CRAF inhibitor may be administered once daily, or twice daily, or three times daily, or four times daily, with the actual dosage and timing of administration determined by criteria such as the patient's age, weight, and gender; the extent and severity of the cancer to be treated; and the judgment of a treating physician.

In one embodiment, Compound A is prepared for oral administration and is administered orally ata dose of 50 mg, 100 mg, 150 mg, 200 mg, 250 mg, 300 mg, 350 mg, 400 mg or 600 mg delivered up to four, e.g., one or two times daily: a dose of 100 mg once or twice daily is projected to provide a plasma concentration in human subjects that could be efficacious in humans based on allometric scaling of corresponding plasma levels in animals, and a dose of 200 mg up to four times daily may be administered to achieve greater efficacy while still providing a satisfactory therapeutic index. In some embodiments, Compound A is administered once daily

(QD) at a dose of 100 mg, 200 mg, 250 mg, 300 mg, 350 mg or 400 mg, or ata dosage of 300 mg, 400 mg or 600 mg twice daily (BID).

In the combinations of the invention, the combination treatments typically use from 50 mg to 1200 mg, typically 50 mg, 100 mg, 200 mg, 250 mg, 300 mg, 350, 400 mg, 600 mg or 1200 mg, more typically 100 mg, 200 mg, 250 mg, 300 mg, 350, 400 mg, 600 mg or 1200 mg, total daily dosage (TTD) of Compound A. The TTD may be administered either QD or BID, Suitably, in the combinations and methods of the invention, a dosage of 100 mg, or 200 mg, or 250 mg, or 350 mg of Compound A is administered once daily ora dosage of 300 mg, 400 mg or 600 mg is administered BID. Preferably the TTD dosage of Compound A in the combinations of the present invention is 800 mg, which may be administered once daily or twice daily.

For example, Compound A may be administered at a total daily dose ranging from 400 mg to 800 mg, administered once daily or twice daily. Typically, Compound A may be administered at a dose of 400 mg BID or 200 mg BID. These doses and dosing regimens of Compound A may be especially beneficial in providing a balance between safety and efficacy in the combinations of the present invention.

In one embodiment, Compound B is prepared for administration via oral delivery, and may be used as its hydrochloride salt. In some embodiments, the compound or its HCl salt is simply encapsulated in a pharmaceutically acceptable container such as a hard or soft gelcap for oral administration. Compound B may be administered at a TTD ranging from 50 to 400 mg. The TTD of Compound B may be administered either QD or BID, preferably QD. Preferably, the TTD of Compound B may be selected from 50 to 300 mg, e.g., from 50, 100, 150, 200, 250 and 300 mg, preferably administered QD, and preferably from 50, 100, 150 and 200 mg, which may be administered QD or BID.

Compound B may be administered in a TTD from 100 mg to about 400 mg (typically from 100 mg to 200 mg (preferably administered once daily). These doses and dosing regimens of Compound B may be especially beneficial in providing a balance between safety and efficacy in the combinations of the present invention.

In one embodiment, Compound A is administered in a TTD from 400 mg to 800 mg (preferably wherein the TTD is administered twice daily, e.g. from 200 mg BID to 400 mg BID) and Compound B is administered in a TTD from 100 mg to 400 mg (typically from 100 mg to 200 mg (preferably administered once daily).

In one embodiment, Compound A is administered in a TDD of 800 mg (e.g., 400 mg twice daily) and Compound B is administered in a TDD of 200 mg (e.g., 200 mg administered once daily).

In one embodiment, Compound A is administered in a TDD of 1200 mg (e.g., 600 mg twice daily) and Compound B is administered in a TDD of 300 mg (e.g. administered once daily).

In one embodiment, Compound A is administered in a TDD of less than 1200 mg (e.g., less than 600 mg twice daily) and Compound B is administered in a TDD of less than300 mg.

In one embodiment, trametinib is prepared for administration via oral delivery, e.g., in the form or a tablet. The unit dosage forms can be produced in a variety of dosages for flexible administration; for example, unit dosage forms, especially tablets, can be prepared containing about 0.5 mg, about 1.0 mg, about 1.5 mg or about 2 mg, e.g., for QD administration. trametinib is preferably administered, preferably once daily, at a total daily dosage of 0.5, 1, 1.5 or 2 mg. Preferably total doses of 0.5 and 1.0 mg of trametinib may be used in the combinations of the invention.

In one embodiment, Compound A is administered in a TTD from 400 mg to 800 mg (preferably wherein the TTD is administered twice daily, e.g. from 200 mg BID to 400 mg BID) and trametinib is administered in a TTD from 0.5 mg to 2 mg (typically from 0.5 mg to 1.0 mg (preferably administered once daily).

In one embodiment, Compound A may be administered in a TDD ranging from 800 mg to 400 mg, e.g., a TDD of 800 mg (e.g., 400 mg administered twice daily) and trametinib may be administered in a TDD of 1 mg (e.g., administered once daily).

In one embodiment, Compound A may be administered in a TDD ranging from 800 mg to 400 mg, e.g., a TDD dose of 800 mg (e.g., 400 mg twice daily) and trametinib may be administered in a TDD of 0.5 mg (e.g., administered once daily).

In one embodiment, Compound A may be administered in a TDD of less than 800mg (e.g., less than 400 mg twice daily) and trametinib may be administered in a TDD of 1 mg, or less than 1 mg.

Compound A plus Compound B or trametinib can be used together according to methods disclosed herein. Two or three of the Compounds can be administered together or separately in any order, depending on the intended dosage amount and frequency of administration, since it is contemplated that the treatments of the invention may be continued for 2 days, 3 days, 4 days, 5 days, 6 days, 1 week, 2 weeks, 3 weeks, 4 weeks, or more than 4 weeks as deemed appropriate to the treating physician, and further as guided using methods described herein to determine a suitable dosage and administration frequency. In the methods and uses disclosed herein, Compound A plus Compound B or trametinib may be administered daily for at least five consecutive days. Typically, all compounds used in a combination according to the invention are administered orally.

The combinations according to the invention can be administered together in a single composition or administered separately in two or more different compositions, e.g.,, compositions or dosage forms as described herein. The pharmaceutical combinations described herein, in particular the pharmaceutical combination of the invention, may be a free combination product, i.e. a combination of two or three or more active ingredients, e.g., Compound A plus Compound

B or trametinib, which is administered simultaneously, separately or sequentially as two or more distinct dosage forms. The administration of the therapeutic agents can be in any order. The first agent and the additional agents (e.g.,, second, third agents) can be administered via the same administration route or via different administration routes.

The NSCLC tumors treated by the pharmaceutical combinations described herein may be at an early, intermediate or advanced state. Especially, treatment with a combination according to the invention is indicated for treatment of NSCLC that does not or does not sufficiently respond to treatment with Compound A alone or Compound B alone or trametinib alone.

The combinations as described herein can be administered to the subject systemically (e.g.,, orally (preferred), parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes.

The combination partners in the dual combination may be administered orally, and may be administered together (at the same time) or separately in any order, following dosing schedules determined by a treating physician; suitable doses and dosing schedules are disclosed herein.

Provided herein is a pharmaceutical combination in the form of a combined preparation comprising (a) one or more dosage units comprising Compound A, or a pharmaceutically acceptable salt thereof, and, in addition, (b) Compound B, or a pharmaceutically acceptable salt thereof, and/or (c) trametinib, or a pharmaceutically acceptable salt thereof, especially also comprising a pharmaceutically acceptable carrier material.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “about” and “approximately” shall generally mean an acceptable degree of error for the quantity measured given the nature or precision of the measurements. Exemplary degrees of error are within 20 percent (%), typically, within 10%, and more typically, within 5% of a given value or range of values. In particular, where a dosage is mentioned as ‘about’ a particular value, it is intended to include a range around the specified value of plus or minus 10%.

In particular, where a dosage is mentioned as ‘about’ a particular value, or a dosate is referred to as a particular value (i.e. without the term “about” preceding that particular value), it is intended to include a range around the specified value of plus or minus 10%, or plus or minus 5%.

As is customary in the art, dosages refer to the amount of the therapeutic agent in its free form. For example, when a dosage of 100 mg of Compound B is referred to, and Compound B is used as its hydrochloride salt, the amount of the therapeutic agent used is equivalent to 100 mg of the free form of Compound B.

The term “combination therapy” or “in combination with” or “co-administered with” refers to the administration of two or more therapeutic agents to treat a BRAF -mutant NSCLC described in the present invention. Such administration encompasses co-administration of the therapeutic agents in a substantially simultaneous manner, or in a sequential manner, either at approximately the same time or at different times. In either case, the treatment regimen will provide beneficial effects of the drug combination in treating the BRAF-mutant NSCLC described herein.

By “a pharmaceutical combination”, “a combination” or “in combination with” or “co-administered with”, it is not intended to imply that the therapy or the therapeutic agents must be physically mixed or administered at the same time and/or formulated for delivery together, although these methods of delivery and corresponding formulations are within the scope described herein. A therapeutic agent in these combinations can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized as single-agent therapeutics.

In embodiments of the invention, the additional therapeutic agent (e.g. the CRAF inhibitor) is administered at a therapeutic or lower-than therapeutic dose relative to a single-agent dose level. In certain embodiments, the concentration of the second therapeutic agent that is required to achieve inhibition, e.g.,, growth inhibition or tumor shrinkage, is lower when the second therapeutic agent (e.g., Compound B or trametinib) is used or administered in combination with the first therapeutic agent (Compound A) than when the second therapeutic agent is administered individually. In certain embodiments, the concentration or dosage of the first therapeutic agent that is required to achieve inhibition, e.g.,, growth inhibition, is lower when the first therapeutic agent is administered in combination with the second therapeutic agent than when the first therapeutic agent is administered individually. In certain embodiments, in a combination therapy, the concentration or dosage of the second therapeutic agent that is required to achieve inhibition, e.g.,, growth inhibition, is lower than the therapeutic dose of the second therapeutic agent as a monotherapy, e.g.,, 10-20%, 20-30%, 30-40%, 40-50%, 50-60%, 60-70%, 70-80%, or 80-90% lower. In certain embodiments, in a combination therapy, the concentration or dosage of the first therapeutic agent that is required to achieve inhibition, e.g.,, growth inhibition, is lower than the therapeutic dose of the first therapeutic agent as a monotherapy, e.g.,, 10-20%, 20-30%, 30-40%, 40-50%, 50-60%, 60-70%, 70-80%, or 80-90% lower.

The term “inhibition” or “inhibitor” includes a reduction in a certain parameter, e.g.,, an activity, of a given molecule or pathway. For example, inhibition of an activity of a targeted kinase (CRAF or ERK 1/2) by 5%, 10%, 20%, 30%, 40% or more is included by this term.

Thus, inhibition can be but need not be 100%.

The term “cancer” refers to NSCLC as defined elsewhere herein. As used herein, the terms “treat”, “treatment” and “treating” refer to the reduction or amelioration of the progression, severity and/or duration of a disorder, e.g.,, a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” -refer to the inhibition of the progression of a proliferative disorder, either physically by, e.g., stabilization of a discernible symptom, physiologically by, e.g.,, stabilization of a physical parameter, or both. In other embodiments the terms “treat”, “treatment” and “treating” refer to the reduction or stabilization of tumor size or cancerous cell count. As used herein, the treatment may be characterized by one of more of: (a) increase of tolerability of the combination therapy (b) maintenance of anti-tumor activity and (c) reduction and or stabilization of tumor size or cancerous cell count.

Thus the dosages and dosage regimens of the present invention may be useful in providing the optimum desired response in a patient suffering from BRAF V600 mutant NSCLC or suffering from BRAF non-V600 mutant NSCLC (e.g.,, a therapeutic response whilst being tolerable and/or have fewer undesired side effects).

In the combinations of the present invention, Compound A may be especially useful when administered at a dose equal to or greater than 300 mg BID, and Compound B may be especially useful when administered at a dose equal to or greater than 100 mg. These doses and dosing regimens have been shown to provide an acceptable balance between efficacy and safety or tolerability.

Pharmaceutical Compositions and Kits

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The pharmaceutical compositions of the invention may include a “therapeutically effective amount” or a “prophylactically effective amount” of a compound of the invention. A “therapeutically effective amount” refers to an amount effective, at dosages and for periods of time necessary, to achieve the desired therapeutic result. A therapeutically effective amount may vary according to factors such as the disease state, age, sex, and weight of the individual. A therapeutically effective amount is also one in which any toxic or detrimental effects of the combination partners are outweighed by the therapeutically beneficial effects. A “therapeutically effective dosage” preferably modulates a measurable parameter in a desired manner, e.g.,, tumor growth rate, by at least about 20%, more preferably by at least about 40%, even more preferably by at least about 60%, and still more preferably by at least about 80% relative to untreated subjects. The ability of a compound to desirably modulate a measurable parameter, e.g.,, cancer, can be evaluated in an animal model system predictive of efficacy in human tumors to help establish suitable dosing levels and schedules. Alternatively, this property of a composition can be evaluated by examining the ability of the compound to modulate an undesired parameter using in vitro assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention is a kit comprising one or more of the Compounds described herein. The kit can also include one or more other elements: instructions for use; other reagents for use with the compound(s); devices or other materials for preparing the compound for administration, such as a mixing container; pharmaceutically acceptable carriers; and devices or other materials for administration to a subject, such as a syringe.

The combinations of the invention have therapeutic or protective functions or both, and can be used in vivo or ex vivo. For example, these molecules can be administered to cells in culture, in vitro or ex vivo, or to a human subject, to treat, prevent, and/or diagnose a variety of disorders, such as cancers as described herein.

Accordingly, in one aspect, the invention provides a method of enhancing the efficacy of an anticancer compound by using it in combination with another anticancer compound, particularly a method using Compound A together with Compound B and/or Compound B to provide enhanced efficacy not safely achievable by administering similar doses of either compound as a single agent. These combinations are particularly useful for treatment of cancers expressing one or more gain of function mutations in the MAPK pathway, particularly mutations in RAS and/or Raf genes.

Where a “subject” is mentioned, this particularly refers to a human, especially in need of NSCLC treatment.

Where “Compound A plus Compound B or trametinib” is referred to, this means a combination of (i) Compound A plus Compound B and (ii) a combination of Compound A plus trametinib.

Further Combination Therapies

In certain embodiments, the methods and compositions described herein are administered in combination with one or more additional other cancer therapy modes such as antibody molecules, chemotherapy, other anti-cancer therapeutic agents (e.g.,, targeted anti-cancer therapies, gene therapy, viral therapy, RNA therapy bone marrow transplantation, nanotherapy, or oncolytic drugs), cytotoxic agents, immune-based therapies (e.g.,, cytokines, immunostimulants, or cell-based immune therapies), surgical procedures (e.g.,, lumpectomy or mastectomy) or radiation procedures, or a combination of any of the foregoing. The additional therapy may be in the form of adjuvant or neoadjuvant therapy. In some embodiments, the additional therapy is an enzymatic inhibitor (e.g.,, a small molecule enzymatic inhibitor) or a metastatic inhibitor. Exemplary cytotoxic agents that can be administered in combination with the combination of the invention include dabrafenib, antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g.,, local or whole body irradiation (e.g.,, gamma irradiation). In other embodiments, the additional therapy is surgery or radiation, or a combination thereof. In other embodiments, the additional therapy is a therapy targeting one or more of PI3K/AKT/mTOR pathway, an HSP90 inhibitor, or a tubulin inhibitor.

Alternatively, or in combination with the aforesaid combinations, the methods and compositions described herein can be administered in combination with one or more of: an immunomodulator (e.g., an activator of a costimulatory molecule or an inhibitor of an inhibitory molecule, e.g.,, an immune checkpoint molecule); a vaccine, e.g.,, a therapeutic cancer vaccine; or other forms of cellular immunotherapy.

Any combination and sequence of other therapeutic agents, procedures or modalities (e.g.,, as described herein) can be used in combination with the treatments of the invention. The compositions and combinations of the invention can be administered before the other treatment methods, concurrently with other treatment methods, between cycles of such other treatments, or during remission of the disorder.

### EXAMPLES

The Examples below are set forth to aid in the understanding of the invention and, though also being specific invention embodiments, are not intended to, and should not be construed to, limit its scope in any way.

### Example 1: Phase Ib study of Compound A+Compound B in patients with KRAS- or BRAF-mutant NSCLC

Compound A is an inhibitor of BRAF/CRAF and Compound B is an inhibitor of ERK1/2. Compound A and Compound B have demonstrated preclinical activity in MAPK-pathway driven xenograft models, alone and in combination. Both have been evaluated as single agents in Phase I dose-finding studies.

Compound A and Compound B combinations were investigated in a Phase Ib, open-label trial (NCT02974725). Eligible patients had advanced/metastatic KRAS- or BRAF-mutant NSCLC. Oral Compound A was administered at 50-350 mg once daily (QD) or 300-600 mg twice daily (BID); oral Compound B was administered at 100-300 mg QD. Objectives were to determine the recommended dose for expansion (RDE) and characterize the safety, pharmacokinetics (PK), and preliminary efficacy of Compound A +Compound B.

Results: Up to the time for which the following results are reported, 49 patients had been treated. 45 (92%) discontinued (primarily due to disease progression, n=29; 59%), 4 were ongoing. Median age was 62 years (range: 35-82), 67% had [symbol] stage IIIB disease, 82% received [symbol] 2 prior therapies. Median duration of exposure to study treatment was 6 weeks (range: 1-36). PK parameters for Compound A+Compound B were consistent with single agent data;

exposure was approximately dose-proportional for both. 5 Dose Limiting Toxicities (DLTs) were reported in 4 patients: Grade (G) 3 rash and G3 hand-foot syndrome; G4 asymptomatic amylase increase (Compound A 600 mg BID+Compound B 100 mg); G3 asymptomatic lipase increase (Compound A 100 mg QD+Compound B 100 mg); G3 retinal detachment (resolved; Compound A 200 mg QD+Compound B 300 mg). Treatment-related AEs were reported in 90% of patients, most commonly ( [symbol] 20%) dermatitis acneiform, nausea (both 29%), pruritis (27%), diarrhea (24%). G3/4 treatment-related AEs were reported in 33% of patients, most commonly ( [symbol] 4%) lipase increase, amylase increase (both 6%), acute polyneuropathy (4%). Maximum tolerated dose was not reached.

Recommended Dose for Expansion (RDE) was declared as Compound A 400 mg BID +Compound B 200 mg QD. 2 patients (4%) had an unconfirmed partial response (uPR) and received treatment >4 months, at Compound A doses [symbol] 300 mg BID. Both were BRAF mutant, one typical (V600E) and one atypical (K601N). 16 patients (33%; including the 2 uPRs) had stable disease (SD), of those, 2 patients with BRAF mutations (V600E and G466A, respectively) had notable tumor shrinkage ( [symbol] 25%). The second19 patients (39%) had progressive disease.

Response was unknown in 13 patients due to discontinuation prior to first assessment and 1 patient due to insufficient data.

Results of the trial hitherto achieved for the two patients with tumor shrinkage can be found in the Table below in the third to last and the last column where LXH254 refers to Compound A, Compound B is an ERK inhibitor, PR means partial remission, Compd. A refers to Compound A, Compd. B refers to Compound B, BOR means Best overall response, SD means stable disease, PD means progressive disease, UNK means unknown, PR means partial response.

| | Compd. | Compd. | Compd. | Compd. | Compd. | Compd. | Compd. | Compd. | Compd. |
|---|---|---|---|---|---|---|---|---|---|
| | A | A | A | A | A | A | A | A | A |
| | 50 mg | 100 mg | 200 mg | 200 mg | 200 mg | 350 mg | 300 mg | 400 mg | 600 mg |
| | QD | QD | QD | QD | QD | QD | BID | BID | BID |
| | Compd. | Compd. | Compd. | Compd. | Compd. | Compd. | Compd. | Compd. | Compd. |
| | B | B | B | B | B | B | B | B | B |
| | 100 mg | 100 mg | 100 mg | 150 mg | 300 mg | 100 mg | 100 mg | 200 mg | 100 mg |
| | QD | QD | QD | QD | QD | QD | QD | QD | QD |
| | N = 5 | N = 4 | N = 6 | N = 5 | N = 7 | N = 5 | N = 5 | N = 6 | N = 6 |
| Number of | 5 | 4 | 6 | 5 | 7 | 5 | 5 | 3 | 6 |
| evaluable | | | | | | | | | |
| patients | | | | | | | | | |

BOR with confirmation - n (%)

| | | | | | | | | | | |
|---|---|---|---|---|---|---|---|---|---|---|
| SD | 1 (20%) | 3 (75%) | 1 | (16.7%) | 3 (60%) | 2 (28.6%) | 1 (20%) | 2 (40%) | 0 | 2 (33.3%) |
| PD | 3 (60%) | 1 (25%) | 3 | (50%) | 1 (20%) | 1 (14.3%) | 3 (60%) | 1 (20%) | 2 (66.7%) | 4 (66.7%) |
| UNK (*) | 1 (20%) | 0 | 2 | (33.4%) | 1 (20%) | 4 (57.1%) | 1 (20%) | 2 (40%) | 1 (33.3%) | 0 |

BOR without confirmation - n (%)

| PR | 0 | 0 | 0 | | 0 | 0 | 0 | 1 (20%) | 0 | 1 (16.7%) |
|---|---|---|---|---|---|---|---|---|---|---|
| SD | 1 (20%) | 3 (75%) | 1 | (16.7%) | 3 (60%) | 2 (28.6%) | 1 (20%) | 1 (20%) | 0 | 1 (16.7%) |
| PD | 3 (60%) | 1 (25%) | 3 | (50%) | 1 (20%) | 1 (14.3%) | 3 (60%) | 1 (20%) | 2 (66.7%) | 4 (66.7%) |
| UNK (*) | 1 (20%) | 0 | 2 | (33.4%) | 1 (20%) | 4 (57.1%) | 1 (20%) | 2 (40%) | 1 (33.3%) | 0 |

(*) All patients with UNK evaluations discontinued before the first RECIST staging

Conclusions: Compound A+Compound B was generally well tolerated and the RDE has been declared. Preliminary signs of efficacy were seen in patients with BRAF-mutant NSCLC. In the combinations of the present invention, Compound A may thus be especially useful when administered at a dose equal to or greater than 300 mg BID, and Compound B may be especially useful when administered at a dose equal to or greater than 100 mg. The RDE is expected to provide a balance between efficacy and safety or tolerability.

### Example 2: Treatment of Patients with BRAF mutant NSCLC with a combination of Compound A and either (i) Compound B or (ii) trametinib.

A study to characterize the safety and tolerability of the dual combination Compound A (LXH254) and Compound B (LTT462) and of the dual combination Compound A (LXH254) and trametinib in patients with non-small cell lung cancer (NSCLC) harboring BRAF mutations is carried out. The combinations may be investigated in patients suffering from BRAF non-V600 mutant NSCLC (e.g. BRAF non-V600E mutant NSCLC) or from a Class I, II or III BRAF-mutant NSCLC (more particularly, NSCLC with Class II BRAF mutations or NSCLC with class III BRAF mutations).

The study treatment is/will be taken until the patient experiences unacceptable toxicity, progressive disease and/or treatment is discontinued at the discretion of the investigator or the patient or due to withdrawal of consent.

Adult NSCLC patients, with confirmed diagnosis of advanced or metastatic KRAS-or BRAF-mutantNSCLC and adult melanoma patients with diagnosis of locally advanced or metastatic NRAS-mutated melanoma who have progressed following standard of care or for whom no effective standard therapy exists, is tolerated, appropriate or is considered equivalent to study treatment are eligible to participate in this study.

Key inclusion criteria:
1. Written informed consent must be obtained prior to any study specific procedures that are not part of standard of care.
2. Age (male or female) 18 years or older at the time of the informed consent.
3. Patients must have advanced or metastatic NSCLC.
4. Presence of BRAF (NSCLC) mutation in tumor tissue prior to study treatment as determined by a local laboratory or a Novartis designated central laboratory, or written documentation of BRAF mutation:
For dose escalation: presence of BRAF mutated NSCLC
For dose expansion: presence of BRAF V600 mutation or BRAF non-V600 mutation (NSCLC).
5. All patients participating in this clinical trial must have progressed following standard therapy or, in the opinion of the Investigator, no effective standard therapy exists, is tolerated, appropriate or is considered equivalent to study treatment.
6. ECOG (Eastern Cooperative Oncology Group) performance status ≤2
7. Patients must have a site of disease amenable to biopsy and must be willing to undergo a new tumor biopsy at baseline and during treatment according to treating institution's own guidelines and requirements for such procedure.
Key exclusion criteria:
1. Dose expansion—For BRAF mutant patients group: Prior treatment with any EGFR, ALK, ROS1, KRAS, RAF (both BRAFV600 selective and pan-RAF), MEK1/2 and/or ERK1/2 inhibitors. (For patients with BRAF V600 mutant NSCLC, prior treatments with BRAF and MEK1/2 inhibitors are allowed).
2. History or current evidence of retinal vein occlusion (RVO) or current risk factors for RVO (e.g. uncontrolled glaucoma or ocular hypertension, history of hyperviscosity or hypercoagulability syndromes).
3. Any medical condition that would, in the investigator's judgment, prevent the patient's participation in the clinical study due to safety concerns or compliance with clinical study procedures. Any severe, acute, or chronic medical or psychiatric condition or laboratory abnormality that may increase the risk associated with study participation or study treatment administration or that may interfere with the interpretation of study results and, in the judgment of the investigator, would make the patient inappropriate for the study.
4. Patients receiving treatment with medications that are known to be strong inhibitors and/or inducers of CYP3A; substrates of CYP2C8, CYP2C9, and CYP3A with a narrow therapeutic index; herb medicines known to cause liver toxicity, which cannot be discontinued 7 days prior to the start study treatment and for the duration of the study.
5. Patients receiving proton pump inhibitors (PPI) which cannot be discontinued 3 days prior to the start study treatment and for the duration of the study.
6. Patients with Gilbert's syndrome or other heritable diseases of bile processing.
Efficacy assessments:

Efficacy may be evaluated measuring overall response rate (ORR), disease control rate (DCR), duration of response (DOR), progression free survival (PFS) as per RECIST version 1.1 and overall survival (OS).

Safety assessments:

Safety may be evaluated measuring incidence and severity of adverse events (AEs) and serious AEs (SAES) including changes in laboratory values, vital signs and ECGs, incidence and nature of DLTs during the first cycle.

Tolerability may be evaluated measuring dose interruptions, reductions and dose intensity.

Compound B, or a pharmaceutically acceptable salt thereof, may be administered in a TTD from 100 mg to 400 mg (typically from 100 mg to 200 mg (preferably administered once daily) and Compound A may be administered in a total daily dose (TTD) from 400 mg to 800 mg (preferably wherein the TTD of Compound A is administered twice daily, e.g. from 200 mg BID to 400 mg BID);

Compound A may be administered in a total daily dose (TTD) from 400 mg to 800 mg (preferably wherein the TTD is administered twice daily, e.g. from 200 mg BID to 400 mg BID) and trametinib may be administered in a TTD from 0.5 mg to 2 mg (typically from 0.5 mg to 1.0 mg (preferably administered once daily).

The clinical trial is still ongoing at the time of writing. Early clinical activity could be observed (with regard to Compound A and Compound B combinations, see Example 1). Additional preliminary results for patients receiving or having received a total daily dose of 800 mg (400 mg administered twice a day) of Compound A and a total daily dose of 200 mg Compound B (200 mg administered once a day) are as follows.

Out of 15 BRAF non-V600 mutant NSCLC, 3 are still ongoing, 12 patients discontinued. 2 out of the three ongoing patients presented a K601E mutation. Out of 3 BRAF V600-mutant NSCLC patients, 2 patients are ongoing and 1 patient discontinued.

### Example 3: Cellular assays with NSCLC mutant cell lines

Cells were plated in 6-well dishes (#3 506, Corning, NY) at a density of 0.5×10⁶ cells per well. One day after plating, lines were treated with low and high concentrations of Compound A (300/3000nM), trametinib (Compound C) (3/30nM) and Compound B (300/3000nM) for 4 and 24 hours. Cells were harvested in RIPA lysis buffer (#89900, Thermo Fisher, Waltham, Mass.) containing protease inhibitors (#87785, Thermo Fisher, Waltham, Mass.) and phosphatase inhibitors (#78420, Thermo Fisher, Waltham, Mass.). Proteins were separated on a 4-12% Bis-Tris NuPAGE SDS gel (#WG1403Bx10, Life Technologies, Carlsbad, Calif.) and transferred to nitro-cellulose using the Trans-Blot Turbo System (Bio-Rad, Hercules, Calif.). Proteins were detected with 1:1000 dilutions of antibodies recognizing pMEK1/2 (#9154, Cell Signaling Technology, Beverly, Mass.), pERK1/2 (#4730, Cell Signaling Technology, Beverly, Mass.), and pRSK (#9348, Cell Signaling Technology, Beverly, Mass.) and 1:5000 dilution of an antibody recognizing β-actin (#AM4302, Life Technologies, Carlsbad, Calif.). Protein levels were detected using anti-mouse-HRP or anti-rabbit-HRP secondary antibodies and developed with SuperSignal West Femto (#34096, Thermo Scientific,Waltham, Mass.) or Dura Chemi-luminescent substrate (#34076, Thermo Scientific,Waltham, Mass.) on a GE Image Quant LAS 4000 imaging system (GE Healthcare, Woburn, Mass.).

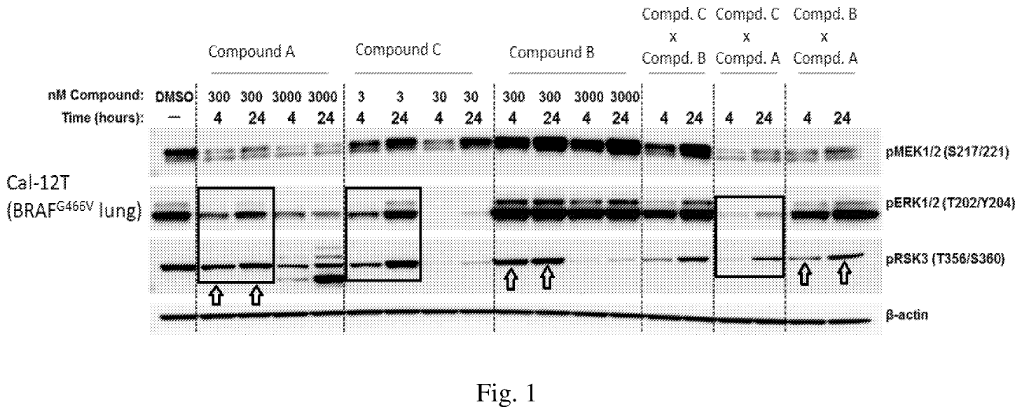

NSCLC cancer -derived cell line CAL-12T, which a class III BRAF variant BRAF^{G466V} (Yao et. al., Nature. 2017 Aug. 10; 548(7666): 234-238), was incubated with low and high concentrations of Compound A (300/3000nM), trametinib (3/30 nM) and Compound B (300/3000nM) for 4 and 24 hours and the inhibition of MAPK signaling, as judged by reductions in phosphorylated MEK, ERK, and RSK determined by western blot analysis as described above. In addition, cells were treated with combinations of each drug in which the lower of the two single agent treatments were combined. Inhibition of signaling by combined low-dose Compound A and trametinib (Compound C) was significantly stronger that comparable single agent treatments, see FIG. 1 (Black framed boxes). Similarly, pathway suppression by combined Compound A and Compound B exceeded that observed for either single agent as judged by levels of phosphorylated RSK3 (arrows).

Compound A in combination with (a) Compound B or (b) trametinib may thus bring additional benefit in treating atypical BRAF-mutant NSCLC and class III BRAF-mutant (e.g.

BRAFG^{466V}-mutant)NSCLC, compared to Compound A, Compound B or trametinib used as single agent,

### Example 4: Activity of Compound A against BRAF mutant cell lines in a xenograft model

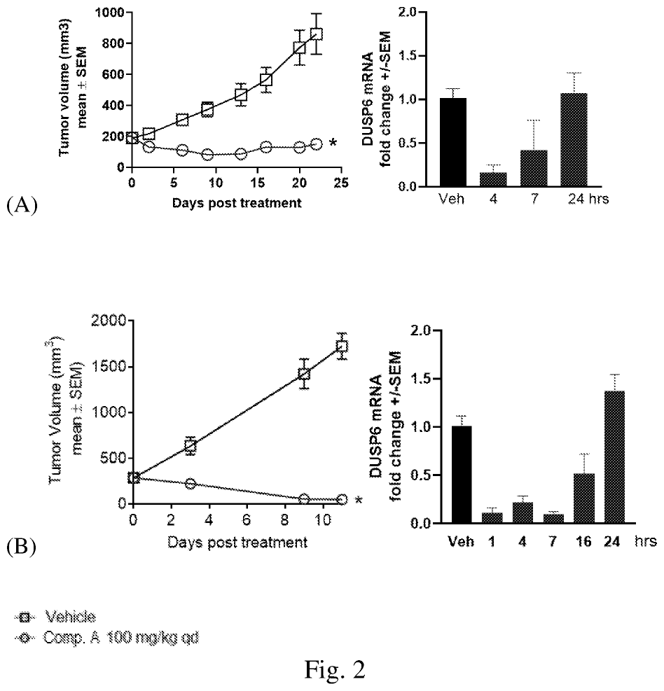

The anti-tumor effects of Compound A in a set of BRAF, NRAS and KRAS mutant xenograft models were investigated. In general, and consistent with cell line sensitivity in vitro, models harboring atypical BRAF mutations co-incident with either mutationally activated NRAS (SK-MEL-30 (BRAF^{D287H}); FIG. 2(A) or KRAS (HEYA8, (BRAF^{G464E}; FIG. 2(B)) either regressed or demonstrated prolonged stasis following treatment with Compound A. RAS mutant tumors, most notably KRAS mutants, were generally less sensitive to Compound A, with most models demonstrating a slow growth phenotype when treated with Compound A (data not shown). Consistent with the anti-tumor results, pathway inhibition in select models mirrored anti-tumor effects (FIG. 2 FIG. 2 (A) and (B). These data indicate that Compound A has robust activity in BRAF-mutant as opposed to RAS-, particularly KRAS-mutant, models.

The following Table shows the experimental data with the two cell lines mentioned with regard to FIG. 2 as just discussed, with an additional cell line, 1855HCCOX (KRAS^{G12V}, BRAF^{D287H})

TABLE
Experimental Data with KRAS and BRAF mutated cell lines

| Model | Cancer | KRAS | NRAS | BRAF | Dose | | |
|---|---|---|---|---|---|---|---|
| Name | Type | variant | variant | variant | (mg/kg) | T/C % | Reg. % |
| 1855HCOX | CRC | G12V | N/A | D287H | 100 (qd) | 15 | nd |
| HeyA8 | OvCa | G12D | N/A | G464E | 100 (qd) | nd | 82 |
| | | | | | 30 (qd) | nd | 75 |
| SK-MEL-30 | Melanoma | N/A | Q61K | D287H | nd | 21 | |

Reg. = Regression
nd not determined
CRC Colorectal Carcinoma
OvCa Ovarial Cancer
N/A Not Applicable

### Experimental:

Mice and statement ofwelfare:

Outbred athymic (nu/nu) female mice (“HSD: Athymic Nude-nu”) (Charles River) and SCID beige (Charles River) mice were allowed to acclimate in the Novartis NIBRI animal facility with access to food and water ad libitum for minimum of 3 days prior to manipulation. Animals were handled in accordance with Novartis ACUC regulations and guidelines.

Cell culture and in vivo efficacy

SK-MEL-30 cell line was purchased from ATCC. HeyA8 cells were obtained from MD Anderson. All cell lines were included in the Novartis Cell Line Encyclopedia (CLE) cell line collection.

All cell lines have been shown to be free of Mycoplasma sp. and murine viruses in the IMPACT VIII PCR assay panel (IDEXX RADIL, IDEXX Laboratories INC, Westbrook, ME).

SK-MEL-30 cells were maintained in RPMI-1640 plus 10% FBS. All FBS was inactivated at 56° C. for 30 min. The SK-MEL-30 cells were kept at 37° C. in a humidified atmosphere containing 5% carbon dioxide. Cells were harvested at 80-95% confluence with 0.25% trypsin-EDTA, washed with PBS and detached with 0.25% trypsin-EDTA, neutralized with growth medium, after centrifugation for 5 min at 1200 rpm. A resuspension of the cell pellet in cold HBSS was mixed with an equal volume of MatrigelTM Matrix to prepare a final concentration of 50×10⁶cells/mL. Then 100ul (5×10⁶ cells) was implanted subcutaneously into the right flank of female nude mice (n=9/group).

HeyA8 cells were cultured in EMEM containing 1% non-essential amino acid and 10% heat-inactivated fetal bovine serum without antibiotics. Cells were harvested at 80-95% confluence, washed, and re-suspended in cold PBS at a concentration of 1×10⁷cells/ml. Finally, 2×10⁶ cells in a total volume of 200 μL were implanted subcutaneously into the upper right flank of nude mice (n=5/group). PS 185 SHCOX tumor xenograft (PDX) tumors where propagated by serial passage of tumor fragments in nude mice. Briefly, 3x3x3 mm fragments of fresh tumor from a previous passage were implanted subcutaneously into the mice. Efficacy testing was carried out with n=6-12/group.

In all cases tumor volume was determined by measurement with calipers and calculated using a modified ellipsoid formula, where tumor volume (TV) was determined using the formula mm³ =[((1×w2)×3.14159))/6], where 1 is the longest axis of the tumor and w is perpendicular to 1. Mice were monitored for tumor growth, body weight and body condition. Animal well-being and behavior, including grooming and ambulation, were monitored twice weekly. General health of mice was monitored daily.

Animals were weighed at dosing day(s) and dose was adjusted according to body weight. Dosing volume was 10 mL/kg. Tumor dimensions and body weights were collected at the time of randomization and twice weekly thereafter for study duration. Animals were sacrificed when tumor volume reached >1000mm³.

Tissue Collection, body weight and tumor volume analysis

Upon sacrifice, tumors were immediately excised and sectioned into 2-3 pieces. Tissue fragments were snap frozen in liquid nitrogen, and stored at −80° C. until processing for PD analysis. Percent change in body weight was calculated as (BW_{curent}-BWᵢₙᵢₜᵢₐₗ)/(BW_{initial)×}100%. Data was presented as mean percent body weight change from the day of treatment initiation ±SEM (not shown). Percent treatment/control (% T/C) values were calculated using the following formula:

% T/C=100×ΔT/ΔC is ΔT≥0

and percent regressions using the formula

% regression=100×ΔT/Tᵢₙᵢₜᵢₐₗ if ΔT<0

where:

T=mean tumor volume of the drug-treated group on the final day of the study;

ΔT=mean tumor volume of the drug-treated group on the final day of the study—mean tumor volume of the drug-treated group on initial day of dosing;

Tᵢₙᵢₜᵢₐₗ=mean tumor volume of the drug-treated group on initial day of dosing;

C=mean tumor volume of the control group on the final day of the study; and

ΔC=mean tumor volume of the control group on the final day of the study—mean tumor volume of the control group on initial day of dosing.

All data in FIG. 2 were expressed as mean±standard error of the mean (SEM). Change in tumor volume and body weight were used for statistical analysis. Between group comparisons were carried out using a one-way ANOVA followed by a Dunnett's multiple comparisons test. For all statistical evaluations, the level of significance was set at p<0.05.

### Drug Formulation

Compound A (free base crystalline form) was dosed p.o. as a solution in MEPC4 vehicle (45% Cremophor RH40+27% PEG400+18% Capmul MCM C8+10% ethanol). Compound A was formulated at 3, 5, and 10 mg/mL.

The data obtained for the cell lines demonstrate that in monogenetically driven tumors benefit by the mentioned treatments is achieved. This supports the view that also in NSCLC cells a benefit by such treatment can be reasonably expected.

Thus Compound A alone and in combination with Compound B or trametinib may be useful in the treatment of BRAF-mutant NSCLC, in particular non -BRAF V600E mutant NSCLC. Compound A alone and in combination with Compound B or trametinib may be useful in the treatment of atypical BRAF-mutant NSCLC.

1. A method for treating non-small cell lung cancer (NSCLC) comprising administering to a patient a pharmaceutical combination comprising:
a CRAF inhibitor which is Compound A, 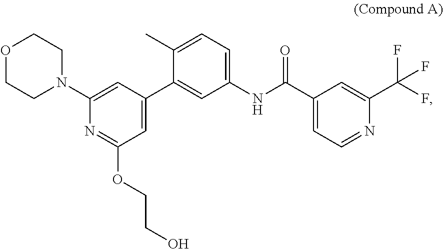 [SMILES: Cc1ccc(NC(=O)c2ccnc(C(F)(F)F)c2)cc1-c1cc(OCCO)nc(N2CCOCC2)c1]
or a pharmaceutically acceptable salt thereof; and
trametinib, or a pharmaceutically acceptable salt or solvate thereof,
wherein the pharmaceutical combination is used in the treatment of a patient with NSCLC with one or more Class II BRAF mutations or NSCLC with one or more class III BRAF mutations.

2. The method according to claim 1, wherein the mutation is selected from V600D, V600K, V600R and V600L.

3. The method according to claim 1, wherein the mutation comprises a class II mutation selected from BRAF kinase domain fusions, specific point mutations and indels which move the ac-helix to an “in” conformation, and wherein the mutation is selected from G469V, G469A, G469L, GV469S, G464V, G464R, E586K, F595L, L597C, L597R, L597S, L595V, A598V, T599I, K601E, K601N, K601 T and A727V.

4. The method according to claim 1, wherein the mutation comprises a class III mutation selected from an F595 mutant, a G596 mutant, G466V, G466R, G466E or G466, S467A, S467E, S467L, G469E, K483M, N581I, N581 S, D594, D594A, D594E, D594G, D594H, D594N or D594V, G596A, G596D, G596F and G596R.

5. The method according to claim 1, wherein the mutation is selected from E26A, V130M and D284E.

6. The method according to claim 1, wherein the mutation comprises two or more of mutations in any combination of Class I, Class II, and Class III.

7. The method according to claim 1, wherein the pharmaceutical combination is suitable for oral administration.

8. The method according to claim 1, wherein Compound A or a pharmaceutically acceptable salt thereof is in an oral dosage form.

9. The method according to claim 1, wherein Compound B or a pharmaceutically acceptable salt thereof is in an oral dosage form.

10. The method according to claim 1, wherein Compound A is administered in a total daily dose (TTD) from 400 mg to 800 mg, wherein the TTD of Compound A is administered in a twice daily (BID) schedule and Compound B is administered in a TTD from 100 mg to 400 mg in a once daily schedule.

11. The method according to claim 1, wherein Compound A is administered at a dose of 400 mg twice daily and Compound B is administered at a dose of 200 mg once daily.

12. The method according to claim 1, wherein Compound A is administered at a dose of 200 mg twice daily and Compound B is administered at a dose of 200 mg once daily.

13. The method according to claim 1, wherein trametinib or a pharmaceutically acceptable salt or solvate thereof is in an oral dosage form.

14. The method according to claim 1, wherein Compound A is administered in a total daily dose (TTD) from 400 mg to 800 mg, wherein the TTD of Compound A is administered in a twice daily (BID) schedule and trametinib is administered in a TTD from 0.5 mg to 2 mg in a once daily schedule.

15. The method according to claim 14, wherein Compound A is administered at a dose of 400 mg twice daily and trametinib is administered ata dose of 0.5 mg once daily.

16. The method according to claim 14, wherein Compound A is administered at a dose of 400 mg twice daily and trametinib is administered ata dose of 1.0 mg once daily.

17.-19. (canceled)

20. The method according to claim 1, wherein Compound A or a pharmaceutically acceptable salt thereof plus Compound B or a pharmaceutically acceptable salt thereof and/or trametinib or a pharmaceutically acceptable salt thereof are administered separately, simultaneously or sequentially.

21.-22. (canceled)

23. The method according to claim 1, wherein the NSCLC harbors a mutation other than RAF mutations V600E, V600D, and G464E, and mutations, A146T, Q61L, Q61K, G12D, G12C, G13D, G12V, and G12R.

24.-31. (canceled)

32. The method according to claim 1, wherein the treatment is characterized by one of more of: (a) increase of tolerability of the combination therapy (b) maintenance of anti-tumor activity and (c) reduction and or stabilization of tumor size or cancerous cell count.
